(19) United States
(12) Patent Application Publication
(10) Pub. No.: US 2006/0051420 A1
(43) Pub. Date: Mar. 9, 2006

(54) Pharmaceutical tablets containing tibolone and a coating

(76) Inventors: Pieter De Haan, Oss (NL); Cornelis Elisabetht Johannus Van Lare, Arnhem (NL)

Correspondence Address: INTERVET INC.;PATENT DEPARTMENT, PO BOX 318, MILLSBORO, DE, 19966-0318, US

(21) Appl. No.: 10/535,009

(22) PCT Filed: Nov. 13, 2003
(86) PCT No.: PCT/EP03/50829

§ 371 Date: May 13, 2005

Related U.S. Application Data
(60) Provisional application No. 60/427,740, filed on Nov. 20, 2002.

(30) Foreign Application Priority Data
Nov. 20, 2002 (EP) 02079920.1

(51) Int. Cl. A61K 9/24; A61K 31/57

(52) U.S. Cl. 424/472; 514/178

(57) ABSTRACT

The present invention relates to a stabilized pharmaceutical tablet comprising an amount of from 0.1 to 10% by weight of tibolone provided with a coating, such as a film coating, a sugar coating, a sugar film coating or a “wrap” coating. The invention further relates to the use of a coating for stabilizing a pharmaceutical tablet comprising tibolone. Preferably, the coating is a sugar coating or a sugar film coating. Preferably, the sugar comprises sucrose. These tablets were shown to have a lower amount of OM38, i.e. an isomerization degradation product of tibolone, after storage as compared to uncoated tibolone tablets.

The present invention relates to a pharmaceutical tablet comprising an amount of from 0.1 to 10% by weight of tibolone.

Compositions comprising tibolone, the chemical name of which is (7α,17α)-17-hydroxy-7-methyl-19-nor-17-pregn-5(10)-en-20-yn-3-one (also denoted as Org OD 14) and a pharmaceutically acceptable solid carrier have been described in EP 389 035.

A known formulation for tibolone is a 100 mg tablet having 2.5 mg of tibolone contained therein, a relatively small amount (e.g., approx. 1% by weight) of pharmaceutically acceptable auxiliaries, and a carrier making up the body of the tablet. The carrier typically is composed of approx. 10% by weight of starch, e.g. potato starch, and approx. 90% by weight of lactose. Tablets of 100 mg containing 2.5 mg of tibolone are available in medical practice under the name of Livial®.

Upon long-term storage of tibolone-containing tablets degradation products of tibolone appear. The major degradation product is (7α,17α)-17-hydroxy-7-methyl-19-nor-17-pregn-4-en-20-yn-3-one (Org OM38). OM38 differs from tibolone in that the double bond in the steroid skeleton is located between positions 4 and 5, whereas in tibolone it is located between positions 5 and 10. This isomerization product is identified as the major impurity in tibolone and in tibolone-containing tablets and limits (at a maximum of 5% by weight of OM38) the approved shelf life of the presently available Livial® tablets to a maximum of two years. Considerable advantage is achieved in prolonging the shelf life of tibolone containing tablets. It is therefore advantageous to find means to reduce the formation of OM38, and to provide more stable tibolone tablets with respect to the amount of OM38 formed after storage.

The problem of reducing OM38 formation in tibolone and tibolone-containing products was addressed earlier in WO 00/23460, providing high purity tibolone containing less than 0.5% by weight of OM38, and in WO 98/47517, providing a composition comprising tibolone and a pharmaceutically acceptable carrier, said carrier having a high starch content, i.e. more than 10%, preferably at least 40% by weight. Despite these contributions to the art, there still is a need to further improve the shelf life of tibolone tablets and for alternatives to the solutions presented in WO 00/23460 and WO 98/47517.

In accordance with the present invention a stabilized pharmaceutical, tablet comprising an amount of from 0.1 to 10% by weight of tibolone provided with a coating is made available for the first time. Surprisingly, such a tablet has a lower content of OM38 after storage, in particular after several months of storage, than a similar tablet without a coating. It is unexpected that a coating reduces the formation of the tibolone-derived isomerisation degradation product OM38.

The present invention further resides in an unexpected new use of a coating for stabilizing a pharmaceutical tablet comprising an amount of from 0.1 to 10% by weight of tibolone.

In the context of the present invention with the term “stabilized” thus is meant “stabilized with respect to the formation of OM38 upon storage.” Hence, the coating of tibolone-containing tablets serves to reduce the formation of OM38 as compared to uncoated tablets. Further, in the context of the present invention the term “tablets” is meant to refer to “solid dosage forms” which can technically be; provided with a coating and which typically are applied for oral administration. Thus, apart from compressed or molded tablets, powders, granules, nonpareils, and capsules are also comprised in the term “tablets.”. Compressed tablets, however, are the most frequently used solid dosage forms.

Incidentally, in WO 98/47517 reference is made to provide tablets of tibolone with a film coat if required (see page 5, lines 14-16). The tablets made in accordance with WO 98/47517 make use of a pharmaceutically acceptable carrier containing more than 10%, preferably at least 40% by weight of starch. In this document, no teaching or suggestion is made with respect to the technical effect of a coating on the formation of OM38 during storage of tibolone-containing tablets. Tibolone tablets stabilized by application of a coating are neither-disclosed nor exemplified in this document. With respect to the words “if required” in line 14 it should be noted that the Livial® tablets that are currently on the market are neither coated nor require a coating with a view to typical reasons for coating, pharmaceutical tablets (taken from Remington, page 894, cited below), i.e. protecting the drug from its surrounding environment (particularly air, moisture, and light), masking of unpleasant taste or odor, increasing the ease by which the product can be ingested by a patient, improving product identity, facilitating handling, improving product appearance, reducing the risk of, interaction between incompatible components, improving product mechanical integrity or modifying drug release. Further, WO 98/47517 only mentions a film coat and makes no reference to any other type of coating.

In the context of the present invention with “coating” is meant any coating which achieves a stabilizing effect with respect to the formation of OM38 upon storage of a coated tablet as compared to an uncoated tablet. Whether or not a coating achieves a stabilizing effect can easily be determined by one skilled in the art on the basis of the present specification. The amount of OM38 present in a tablet is determined via HPLC analysis in a conventional way.

Suitable examples of coatings that can be used in accordance with the present invention include a film coating, a sugar coating, a sugar film coating for example, a sugar film coating according to U.S. 2002/0044970, and a wrap coating according to U.S. Pat. No. 5,146,730. The compositions of these coatings and the methods and equipment by which they are applied onto tablets are well-known to a person skilled in the art and they are described, for example, by Stuart. C. Porter in Chapter 46: Coating of pharmaceutical dosage forms in Remington: The Science and Practice of Pharmacy; 20th ed., 2000; Alfonso R. Gennaro, Editor, Lippincott Williams & Wilkins; Baltimore, USA, as well as in U.S. 2002/0044970 and U.S. Pat. No. 5,146,730. Preference is given to aqueous coating compositions, including mixtures of water and an organic solvent such as an alcohol. Most preferably, coating compositions containing water as the only solvent or vehicle are used.

In particular, the coating to be used in accordance with the present invention is a sugar coating, a sugar film coating or a “wrap” coating. Particularly, the coating of the present invention is a film coating, a sugar coating or a sugar film coating. In a preferred embodiment of the present invention the coating is a sugar coating or a sugar film coating. The sugar coating may be used with or without a seal coat and/or subcoat.

With respect to a film coating, the present inventors have found that not all film coatings give the desired stabilizing effect on tibolone tablets. In particular, tibolone tablets coated with Eudragit® E PO and Eudragit® L100, which are acrylic polymer-containing coatings used for moisture protection, taste and odor masking and for drug delivery in the jejenum, respectively, showed a higher content of OM38 after storage as compared to uncoated tablets. Accordingly, these film coating should not be used.

As explained in Chapter 46 of Remington (cited above), the major components in any film coatings formulation usually consist of a polymer, a plasticizer, a colorant, and a solvent. In accordance with the present invention, a plasticizer and a colorant are optional ingredients and—as stated above—the solvent or vehicle preferably is an aqueous solvent or vehicle or most preferably it is water.

Suitable examples of polymers include cellulose ethers, such as hydroxypropyl cellulose, methylcellulose, and ethylcellulose, and particularly hydroxypropyl methylcellulose. Suitable cellulose ether-containing coating compositions are commercially available under the name of Sepifilm™, Opadry®, and Aquacoat®. Acrylic polymers such as methacrylate and methyl methacrylate copolymers, vinyl polymers such as polyvinyl alcohol and polyvinyl pyrrolidone, gelatin or starches, may also be used. Suitable acrylic polymer-containing coating compositions are commercially available under the name of Eudragit®. Suitable vinyl polymer-containing coating compositions are commercially available under the name of Opaglos®, Opalux™, and Opadry®. In particular in accordance with the present invention use is made of a cellulose ether, a vinyl polymer or gelatin, more in particular a cellulose ether or a vinyl polymer.

Typical plasticizers include glycerin (or glycerol), propylene glycol, polyethylene glycol, triacetin (or glyceryl triacetate), acetylated monoglyceride, citrate esters (e.g. triethyl citrate), and phthalate esters (e.g. diethyl phthalate). Preferred plasticizers are glycerin or glycerol, propylene glycol, and polyethylene glycol.

As explained in Chapter 46 of Remington (cited above), a sugar coating typically consists of a seal coating or sealing, a subcoating, and a sugar coating. In said latter sugar coating a polymer typically is not present. In accordance with the present invention the seal coating and the subcoating are optional coatings and need not be used in combination with a sugar coating in order to obtain the technical effect of the present invention. In particular, the stabilizing effect on OM38 formation can already be observed in sugar coated tablets without a seal coat and/or subcoat.

The sugar used in a sugar coating typically is sucrose (or saccharose), but other saccharides such as glucose, fructose, and lactose or polyalcohols such as (sorbitol, mannitol, and xylitol may also be used in accordance with the present invention. Either one or more sugars may be used in accordance with the invention. Preferably, the sugar comprises sucrose.

A sugar film coating is a coating comprising a polymer and a sugar as defined above. For example, the sugar film coating according to U.S. 2002/0044970 makes use of an aqueous sugar coating liquid containing 30-54% by weight of saccharides (preferably sucrose), 2-10% by weight of polyethylene glycol, and 0.2-2% by weight of polyvinyl pyrrolidone, giving rise to a mono-layered sugar coated tablet JP 2001026534 describes a sugar film coat comprising sucrose, pullulan, and hydroxypropyl methylcellulose. Based on this, a person skilled in the art will find no trouble in making suitable variations in a sugar film coating.

The “wrap” coating according to U.S. Pat. No. 5,146,730 typically makes use of a water-based gelatin preparation having about 45% by weight of gelatin and about 9% by weight of a plasticizer (glycerin and/or sorbitol). With the method of U.S. Pat. No. 5,146,730, tablets are enrobed or “wrapped” in a gelatin coating formed by application of respective layers of elastic gelatin film to opposite sides of the tablet. The applied gelatin layers conform tightly to the tablet surface, bond securely to the tablet, and are sealed together in essentially edge-to-edge manner at a seal line which extends around the tablet at a desired place on the tablet. The coating is applied by feeding a tablet into a cavity formed between a pair of dies over two elastic self-adherent films. A tablet, which is enveloped in this manner between the films is finally coated by sealing the films to each other along the lines contiguous to the tablet. Instead of gelatin, (modified) starches, alginates, modified gelatin, acrylates, polyvinyl pyrrolidone, cellulose derivatives both esters and ethers, and polysiloxanes may also be used. The details of this method and of the “wrap” coating composition are described in U.S. Pat. No. 5,146,730.

The coating to be used in accordance with the present invention typically also contains pharmaceutically acceptable auxiliaries such as arabic gum (or acacia), calcium carbonate, magnesium stearate, talc, china clay, and/or titanium dioxide. Such auxiliaries may be used in conventional quantities if desired. A colorant may also be used if desired.

As is known to the person skilled in the art of coating and as exemplified in the examples below, either one or more than one coating layers may be used and if more than one layer is used, the layers may be of the same or different composition. Particularly, in accordance with the present invention a single layer coating is used.

If desired, the coated pharmaceutical tablet of the present invention can be subjected to smoothing, polishing or printing as is known in the art.

The pharmaceutical tablets to be used in accordance with the present invention may vary in weight and/or in tibolone content. Particularly, 100 mg tablets containing 2.5 mg of tibolone (i.e. 2.5% by weight) are currently on the market and 65 mg tablets containing 1.25 mg of tibolone (i.e. 1.9% by weight) are currently under investigation. Results on both are provided in the examples given below. The pharmaceutically acceptable carrier may vary in composition and be composed of approx. 10% by weight of starch and approx. 90% by weight of lactose, or contain higher amounts of starch as described in WO 98/47517. In particular, the carrier contains less than 40%, more in particular 10% or less by weight of starch. In a preferred embodiment of the invention, stabilized coated Livial® tablets are provided.

The present invention is illustrated by the following Examples.

### EXAMPLE 1

Tablets of 65 mg total weight, containing 1.25 mg of tibolone, were prepared from purified tibolone. The latter was obtained according to the method described in WO 00/23460.

A basic granulate, consisting of 10% potato starch and 90% lactose, was manufactured in a Fluid Bed Granulator, using a starch mucilage as binding liquid.

Uncoated Tibolone Tablets

65 mg tablets having the composition: 1.25 mg tibolone, 63.29 mg basic granulate, 0.13 mg ascorbyl palmitate, and 0.33 mg magnesium stearate, were prepared as follows: approximately 10% of the basic granulate was premixed with tibolone and ascorbyl palmitate. After screening the premix through a 250 μm sieve, the rest of the basic granulate was added and mixing was continued. Finally, magnesium stearate was admixed and the final mixture was tabletted to tablets with a diameter of 5 mm.

Film Coating

Tablets prepared as described above were provided with a film coat (1.2 mg per tablet) having the following composition: 0.75 mg hydroxypropyl methyl cellulose E15, 0.15 mg polyethylene glycol 400, 0.11 mg titanium dioxide, and 0.19 mg talc. An aqueous dispersion of the coating excipients was sprayed onto the tablets using standard film coating equipment (Accela Cota™ 24″) at a rotation speed of the coating pan of 12.5 rpm, an inlet temperature of approx. 60° C., and an airflow of approx. 300 m³/hour.

Sugar Coating I

A batch of approx. 16 kg of tablets film coated as described above (now having the function of a seal coat) was further provided with a sugar-containing subcoat, a sugar-containing layering powder, and a sugar coat.

For application of the coats, tablets were added to a sugar coat pan. The rotating pan had a diameter of about 0.7 m and a rotation speed of 42 rpm. Room conditions were 21° C. and 46% relative humidity.

A subcoat consisting of an aqueous 60.2 w/w % dispersion of approx. 20% arabic gum and approx. 80% saccharose was added manually to the tablets in the sugar coat pan step by step. After each addition, a total of a few hundred grams of a layering powder consisting of 64.3% talc, 21.4% calcium carbonate, 7.1% saccharose, and 7.1% titanium dioxide was added manually. Between the addition of subcoat and layering powder, the tablets were dried in open air without forced drying. After finalizing the subcoat (subcoat and layering powder approx. 26 mg per tablet), a sugar coat dispersion with the composition indicated in Table 1 was added in a number of sequential steps and finally the batch of sugar coated tablets was dried (sugar coat approx. 39 mg per tablet).

TABLE 1

| | |
|---|---|
| Arabic gum | 3.4 |
| Saccharose | 43.6 |
| Calcium carbonate | 6.7 |
| Talc | 10.7 |
| Titanium dioxide | 4.7 |
| Polyethylene glycol | 0.7 |
| Glucose syrup | 0.7 |
| Glycerol 86.5% | 0.1 |
| China clay | 6.7 |
| Purified water | 22.7 |

The amount of the components are provided in weight percentages of the total weight of the composition.

Sugar Coating II

A batch of 8 kg of tablets film coated as described above was provided with a sugar coat, but now without a subcoat, as described above.

Tablets prepared and coated as described above were packed in open brown glass bottles and were subjected to the storage condition of 25° C. and 60% relative humidity (RH) or 40° C. and ambient relative humidity.

After six months, the amount of the isomerization degradation product OM38 was measured in the tablets via HPLC analysis as a percentage of the declared amount of tibolone in the tablets. The results are depicted in Table 2.

TABLE 2

| | |
|---|---|
| At 25° C., | At 40° C., |
| 60% RH¹⁾ | ambient RH |

| | % OM38 | SD²⁾ | % OM38 | SD²⁾ |
|---|---|---|---|---|
| Uncoated tablets | 2.83 | 0.02 | 6.53 | 0.02 |
| Film coated | 2.45 | 0.03 | 4.83 | 0.09 |
| Sugar coated I | 2.32 | 0.02 | 3.19 | 0.04 |
| Sugar coated II | 2.39 | 0.01 | 3.44 | 0.05 |

¹⁾RH is Relative Humidity of the environment.
²⁾SD is the standard deviation in the mean obtained from three measurements from pooled tablets of 10 tablets per pool.

The results in Table 2 show that the coating of tibolone tablets in accordance with the present invention results in a decrease in the formation of the isomerization degradation product OM38 and thus leads to stabilized tibolone tablets. Sugar coating gave better results than just film coating.

### EXAMPLE 2

Sugar Coating

A batch of approx. 20 kg of uncoated tablets (65 mg, see Example 1) was provided with a sugar coat.

For application of the coat, tablets were added to a conventional film coat pan equipped with an air bypass (Dumoulin IDA 30×). The rotating pan had a diameter of about 1 m and a rotation speed of 3 to 5 rpm.

A sugar coat dispersion with the composition indicated in Table 3 was added in a number of sequential steps until a sugar coat of approx. 35 mg per tablet was reached and finally the batch of sugar coated tablets was dried and analysed as (described in Example 1. The results after two months of storage are depicted in Table 4.

TABLE 3

| | |
|---|---|
| Arabic gum | 1.8 |
| Saccharose | 59.5 |
| Talc | 3.0 |
| Titanium dioxide | 0.9 |
| Sunset yellow | 0.002 |
| Purified water | 34.8 |
| Total | 100 |
| Carnauba wax¹⁾ | 0.1 |

¹⁾Added after the application of the coat to obtain a shiny appearance

The amount of the components are provided in weight percentages of the total weight of the composition.

TABLE 4

At 40° C.,
ambient RH

| | % OM38 | SD¹⁾ |
|---|---|---|
| Uncoated tablets | 2.65 | 0.09 |
| Sugar coated | 1.66 | 0.02 |

¹⁾SD is the standard deviation in the mean obtained from three measurements from pooled tablets of 10 tablets per pool.

The results in Table 4 show that the coating of tibolone tablets in accordance with the present invention results in a decrease in the formation of the isomerization degradation product OM38 and thus leads to stabilized tibolone tablets.

### EXAMPLE 3

Tablets of 100 mg total weight, containing 2.5 mg of tibolone, were prepared following the procedure described in Example 1.

Uncoated Tibolone Tablets

100 mg tablets with a diameter of 6 mm having the composition: 2.5 mg tibolone, 96.8 mg basic granulate, 0.2 mg ascorbyl palmitate, and 0.5 mg magnesium stearate, were prepared as described in Example 1.

Film Coating

Tablets prepared as described above were provided with a film coat (1.51 mg per tablet) having the following composition: 0.94 mg hydroxypropyl methyl cellulose E15, 0.19 mg polyethylene glycol 400, 0.14 mg titanium dioxide, and 0.24 mg talc. An aqueous dispersion of the coating excipients was sprayed onto the tablets using standard film coating equipment (Accela Cota™ 24″) at a rotation speed of the coating pan of 12.5 rpm, an inlet temperature of approx. 60° C., and an airflow of approx. 300 m³/hour.

Sugar Coating

A batch of 14 kg of tablets film coated as described above was provided with a sugar coat in the same manner as described in ‘Sugar coat I’ of Example 1 (approx. 35 mg per tablet of subcoat and layering powder and approx. 8 mg per tablet of sugar coat).

The tablets were analysed as described in Example 1 and the results after six months of storage are depicted in Table 5.

TABLE 5

| | At 25° C., 60% RH¹⁾ | At 40° C., ambient RH |
|---|---|---|
| | % OM38 | % OM38 |
| Uncoated tablets | 3.07 | 6.05 |
| Film coated | 2.74 | 4.88 |
| Sugar coated | 2.72 | 3.83 |

¹⁾RH is Relative Humidity of the environment.

The results in Table 5 show that the coating of tibolone tablets in accordance with the present invention results in a decrease in the formation of the isomerization degradation product OM38 and thus leads to stabilized tibolone tablets.

### EXAMPLE 4

Tablets of 65 mg total weight, containing 0.625 mg of tibolone, were prepared following the procedure described in Example 1.

Uncoated Tibolone Tablets

65 mg tablets having the composition: 0.625 mg tibolone, 63.9 mg basic granulate, 0.13 mg ascorbyl palmitate, and 0.325 mg magnesium stearate, were prepared as described in Example 1.

Film Coating I

Tablets prepared as described above were provided with a film coat having the following composition: 5.2 mg Opadry® AMB, consisting of polyvinyl alcohol, titanium dioxide, talc, iron oxide yellow, lecithin, xanthan gum and iron oxide red. An aqueous dispersion (solids content 15 w/w %) of the coating excipients was sprayed onto the tablets using standard film coating equipment (Glatt® GC300) at a rotation speed of the coating pan of approx. 15 rpm, an inlet temperature of approx. 60° C., and an airflow of approx. 100 m³/hour.

Film Coating II

Tablets prepared as described above were provided with a film coat having the following composition: 5.2 mg Sepifilm™ LP770, consisting of hydroxypropyl methylcellulose, stearic acid, and talc. An aqueous dispersion (solids content 8 w/w %) of the coating excipients was sprayed onto the tablets using standard film coating equipment (Glatt® GC300) at a rotation speed of the coating pan of approx. 15 rpm, an inlet temperature of approx. 60° C., and an airflow of approx. 100 m³/hour.

The tablets were analysed as described in Example 1 and the results after six months of storage are depicted in Table 6.

TABLE 6

| | |
|---|---|
| At 25° C., | At 40° C., |
| 60% RH¹⁾ | 40% RH |

| | % OM38 | SD²⁾ | % OM38 | SD²⁾ |
|---|---|---|---|---|
| Uncoated tablets | 4.84 | 0.08 | 17.10 | 0.60 |
| Film coated I | 3.57 | 0.02 | 10.69 | 0.14 |
| Film coated II | 3.51 | 0.06 | 5.77 | 0.05 |

¹⁾RH is Relative Humidity of the environment.
²⁾SD is the standard deviation in the mean obtained from three measurements from pooled tablets of 10 tablets per pool.

The results in Table 6 show that the coating of tibolone tablets in accordance with the present invention results in a decrease in the formation of the isomerization degradation product OM38 and thus leads to stabilized tibolone tablets.

### EXAMPLE 5

Uncoated Tibolone Tablets

65 mg tablets with a diameter of 5 mm having the composition: 0.644 mg tibolone, 63.9 mg basic granulate, 0.13 mg ascorbyl palmitate, and 0.325 mg magnesium stearate, were prepared following the procedure described in Example 1.

Film Coating I

Tablets prepared as described above were provided with 5.2 mg per tablet of a film coat having the following composition: 3.07 mg Eudragit® E PO, 0.31 mg sodium lauryl sulfate, 0.78 mg stearic acid, and 1.04 mg magnesium stearate. An aqueous dispersion (solids content 16 w/w %) of the coating excipients was sprayed onto the tablets using standard film coating equipment (Glatt® GC300) at a rotation speed of the coating pan of approx. 15 rpm, an inlet temperature of approx. 45° C., and an airflow of approx. 100 m³/hour.

Film Coating II

Tablets prepared as described above were provided with 5.2 mg per tablet of a film coat using an aqueous film coat dispersion having the following composition (in w/w % of the total dispersion): 11.1% Eudragit® L 100, 5.6 triethy citrate, 3.7% 1N NH₃, 74% purified water, and 5.6% talc. The aqueous dispersion was sprayed onto the tablets using standard film coating equipment (Glatt® GC300) at a rotation speed of the coating pan of approx. 20 rpm, an inlet temperature of approx. 45° C., and an airflow of approx. 80 m³/hour.

Film Coating III

Tablets prepared as described above were provided with 5.2 mg per tablet of a film coat using an aqueous film coat dispersion having the following composition (in w/w % of the total dispersion): 39.2% Eudragit® RL 30D, 2.4% triethyl citrate, 52.5% purified water, and 5.9% talc. The aqueous dispersion was sprayed onto (the tablets using standard film coating equipment (Glatt® GC300) at a rotation speed of the coating pan of approx. 15 rpm, an inlet temperature of approx. 45° C., and an airflow of approx. 80 m³/hour.

Film Coating IV

Tablets prepared as described above were provided with 5.2 mg per tablet of a film coat using an aqueous film coat dispersion having the following composition (in w/w % of the total dispersion): 41.7% Eudragit® NE 30D, 45.8% purified water, and 12.5% talc. The aqueous dispersion was sprayed onto the tablets using standard film coating equipment (Glatt® GC300) at a rotation speed of the coating pan of approx. 20 rpm, an inlet temperature of approx. 45° C., and an airflow of approx. 80 m³/hour.

The tablets were analysed as described in Example 1 and the results after two months of storage are depicted in Table 7.

TABLE 7

| | |
|---|---|
| At 25° C., | At 40° C., |
| 60% RH¹⁾ | 40% RH |

| | % OM38 | SD²⁾ | % OM38 | SD²⁾ |
|---|---|---|---|---|
| Uncoated tablets | 1.72 | 0.01 | 4.27 | 0.02 |
| Film coated I | 2.65 | 0.03 | 6.71 | 0.07 |
| Film coated II | 3.05 | 0.06 | 8.89 | 0.33 |
| Film coated III | 1.27 | 0.01 | 4.06 | 0.05 |
| Filmcoated IV | 1.70 | 0.02 | 4.86 | 0.02 |

¹⁾RH is Relative Humidity of the environment.
²⁾SD is the standard deviation in the mean obtained from three measurements from pooled tablets of 10 tablets per pool.

The results in Table 7 show that not all coatings of tibolone tablets result in a decrease in the formation of the isomerization degradation product OM38, in particular when using Eudragit® E PO or Eudragit® L 100 worse results were obtained as compared to uncoated tablets.

1. A stabilized pharmaceutical tablet comprising an amount of from 0.1 to 10% by weight of tibolone provided with a coating.

2. The tablet according to claim 1, wherein the coating is a film coating, a sugar coating, a sugar film coating or a “wrap” coating.

3. The tablet according to claim 2, wherein the coating is a sugar coating or a sugar film coating.

4. The tablet according to claim 1, wherein the film coating contains a cellulose ether, a vinyl polymer or gelatin.

5. The tablet according to claim 3, wherein the sugar comprises sucrose.

6. The tablet according to claim 1, wherein the coating contains a plasticizer selected from the group consisting of glycerin, propylene glycol, and polyethylene glycol.

7. A method of stabilizing a pharmaceutical tablet comprising an amount of from 0.1 to 10% by weight of tibolone, comprising:
applying a coating to said tablet.

8. The method according to claim 7, wherein the coating is a film coating, a sugar coating, a sugar film coating or a “wrap” coating.

9. The method according to claim 8, wherein the coating is a sugar coating or a sugar film coating.

10. The method according to claim 9, wherein the sugar comprises sucrose.
